(19) United States
(12) Patent Application Publication
(10) Pub. No.: US 2006/0161216 A1
(43) Pub. Date: Jul. 20, 2006

(54) Device for neuromuscular peripheral body stimulation and electrical stimulation (ES) for wound healing using RF energy harvesting

(76) Inventors: Constance M. John, San Francisco, CA (US); Varghese John, San Francisco, CA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/253,905

(22) Filed: Oct. 18, 2005

Related U.S. Application Data
(60) Provisional application No. 60/619,757, filed on Oct. 18, 2004.

(51) Int. Cl. A61N 1/18

(52) U.S. Cl. 607/40; 607/48

(57) ABSTRACT

A device for electrical stimulation using radio frequency harvesting is disclosed. The device called the BioMed chip is implantable in a patient, the chip circuit comprising a radio frequency harvesting and power circuit and a stimulation circuit, and a plurality of electrodes coupled to the circuit, the plurality of electrodes providing stimulation to targeted areas of the human body. The electrodes may provide stimulation to targeted areas of the body including deep within the body.

### CROSS REFERENCE TO RELATED APPLICATIONS

The present application claims priority under 35 U.S.C. 119(e) from provisional patent application Ser. No. 60/619,757, entitled “Device For Neuromuscular Stimulation (NMS) and Electrical Stimulation (ES) for wound healing using RF energy harvesting”, filed on Oct. 18, 2004, the disclosure of which is herein incorporated by reference in its entirety.

### FIELD OF THE INVENTION

The present invention relates generally to systems and apparatus for providing electrical stimulation and more particularly to a device or a chain of devices for harvesting radio frequency (RF) energy that can be implanted in the human body to produce electrical stimulation in different regions of the body. The device of the invention could be a chip or a nano or molecular system.

### BACKGROUND OF THE INVENTION

Synchronized neuromuscular stimulation (SNMS) encompasses conditions in which unilateral paralysis can be restored by using a RF signal to coordinate the contraction between a functional and paralytic muscle. One application is facial nerve paralysis, often due to Bell's palsy. About 80,000 Americans a year are diagnosed with Bell's palsy and of those, 10% to 15% are left with permanent visible defects such as ptosis of one side of the face. Currently there is no device that can achieve synchronized neurostimulation. However preliminary results of use of RF harvesting has been published in the following article (“Communication between Functional and Denervated Muscles using Radio Frequency” authored by Doreen Jacob and Dr. Marlin Mickle in the Journal—Otolaryngology-Head and Neck Surgery, 2005)

An exciting area for the application of neurostimulation is Vagus Nerve Stimulation (VNS). Cyberonics recently has received FDA approval for the use of the VNS device for the treatment of Refractory Depression. The current VNS device consists of a pace maker that is conected to electrodes on the vagus nerve. The Pace maker and battery are surgically implanted in the chest. (J. Clin. Psychiatry, (2005), 66, 1097-1104; Hanforth A, et. al. Neurology, (1998), 51, 48-55) In addition VNS is being evaluated for Alzhiemer's Disease (AD) and for refractory epilepsy. Conventional medication becomes refractory in certain patients these patients would be most benefited by such treatments. The Vagus Nerve Stimulation (VNS) Therapy treating Alzheimer's disease (AD) has been published in the November issue of the Journal of Clinical Psychiatry. The article, entitled “Cognition-Enhancing Effect of Vagus Nerve Stimulation in Patients with Alzheimer's disease: A Pilot Study” suggests that VNS Therapy may improve cognition in patients with AD (Sjogren MJ, et. al., J Clin. Psychiatry, (2002), 63, 972-980.)

In this study ten patients with mild to moderate AD were implanted with the VNS Therapy system and stimulated with left cervical (neck area) vagus nerve stimulation in the same way the treatment is currently delivered to treat patients with pharmacoresistant epilepsy. Response was defined as an improvement in or no worsening of AD symptoms based on the Alzheimer's disease Assessment Scale-cognitive (ADAS-cog). Patients with AD typically worsen nine points in the ADAS-cog each year. After three months of VNS Therapy, seven of ten patients with AD responded to VNS Therapy according to the ADAS-cog. Of the seven responders, six had improvements in their cognitive symptoms of AD and one had no worsening. The median change on the ADAS-cog was a 3 point improvement. After six months of VNS Therapy, seven of the ten patients had improvements in symptoms compared with their baseline ADAS-cog assessments. The ten patients had a median improvement in the ADAS-cog of 2.5 points after six months of VNS Therapy.

Gastric electrical stimulation (GES) is a technique involving electrical stimulation of the stomach for the treatment for obesity or gastroparesis. The current device involves use of a pace maker with electrodes going to the stomach. Transneuronix pioneered the technique and demonstrated that the technique has affects on obese patients. The pacing was described as 24 hours a day, 180-400 ms pulse width, 40-100 Hz; 2 seconds ON, 3 seconds OFF with a burst amplitude of 3-8.5 mA (Cigaina et.al., Obes Surg. 1999;9:333-334).

Localized drug delivery provides delivery of small amounts of drugs, growth factors, or plasmids such as for gene therapy in a localized and sustained manner and would be of great benefit for a number of applications such as tissue repair, bone regeneration, or rheumatoid arthritis. The inability to deliver growth factors locally in a transient but sustained manner is a substantial barrier to tissue regeneration. Systems capable of localized plasmid gene delivery for prolonged times may offer lower toxicity and should be well-suited for gene therapy and growth factor therapeutics (Nature Medicine 5, 753-759 (1999).

A prior art embodiment is shown in FIG. 1 which consists of a pace maker device that is implanted in the chest and with leads going to the Vagus nerve or to the stomach for gastric electrical stimulation (Lin Z, Dig Dis Sci., 50, 1328-1334, 2005).

Prior art for wound healing is the standard band aid. However it has been shown that certain wounds heals more rapidly in the presence of a mild electrical stimulation. The reason for the rapid healing is the recruitment of macrophages and other tissue repair mechanism by the mild electrical stimulation. It is difficult to administer such a mild electrical stimulation to wounds (Baker, LL, Diabetes Care, Vol 20, 3, 405-412 (1997).

The prior art for current implantable devices for peripheral body stimulation has several limitations that can lead to adverse effects including breakage of leads and need for battery replacement.

Implantation of the conventional devices in the pheriphery is costly. As per FIG.1 the implantation of the electrode 100 and the implantable pulse generator 120 is sometimes performed on different days. The incisions can be prone to infection in the immediate postoperative period. In some elderly patients with thin skin, the pulse generator 110 or wire can erode through the skin and become exposed to potential contamination. Infection or erosion often results in the need to remove the entire device, as antibiotic treatment alone in this setting rarely will clear the infection adequately. The lead 110 restricts the patient's mobility and may break. Furthermore, the battery 120 must be replaced every three to five years. Additional drawbacks of the pacemaker device include the risk of erosion of the leads or hardware, infection, and magnetic sensitivity.

In order to obviate the need for long leads and batteries, attempts have been made in the prior art to transmit energy through space from a base station to a remote station. One such system is disclosed in U.S. Pat. No. 6,289,237 entitled “Apparatus for Energizing a Remote Station and Related Method”. The base station transmits energy which may be RF power, light, acoustic, magnetic or other suitable forms of space transmitted or “radiant” energy to the remote station. Within the remote station, the received energy is converted into DC power which serves to operate the remote station. The source of power for the remote station is the base station and, therefore, there is no need for the remote station to carry an electrical storage device such as a battery. It is suggested that this facilitates the remote station being encapsulated within a suitable protective material, such as a resinous plastic. Homopolymers, elastomers and silicon dioxide are also suggested as suitable materials for such purposes. Further, it is suggested that this facilitates miniaturization of the remote station and placing the remote station in finctionally desirable locations which need not be readily accessible. The remote station, for example, could be implanted in a patient.

The use of a wireless communication link between a base station and transponders in a radio frequency identification system employing modulated back-scattered waves is also known. See Rao, An Overview of Bulk Scattered Radio Frequency Identification System (RFID) IEEE (1999). It has also been suggested to employ a silicon chip in a transponder having a change pump on voltage doubler current. Hornby, RFID Solutions for the Express Parcel and Airline Baggage Industry, Texas Instruments, Limited (Oct. 7, 1999).

For use in miniaturized electronic chip systems, an electronic article containing a microchip having at least one antenna structured to communicate with an antenna remotely disposed with respect to the microchip is disclosed in U.S. Pat. No. 6,615,074 entitled “Apparatus for Energizing a Remote Station and Related Method”. Power enhancement is achieved using a voltage doubler. The antenna of the disclosed apparatus is comparable in volume to a Smart Dust device. Smart Dust is a combination MEMS/Electronic device on the order of 1 mm×1 mm×1 mm.

What is needed therefore is a stimulation device that overcomes the disadvantages of the prior art stimulation devices. What is needed is a stimulation device that requires a single implantation site and surgery. What is also needed is a stimulation device that uses RF energy as a power source. What is further needed is a stimulation device that converts RF energy and stores the converted RF energy. What is also needed is stimulation device that is flexible and implantable anwhere in the body. What is needed is a stimulation device that does not require leads or a pulse generator to be placed outside of the body that is subject to disconnection or breakage. What is also needed is a stimulation device for electrical stimulation that is smaller and more self-contained and that does not require a pulse generator to be implanted elsewhere in the body. What is further needed is a device that can be designed as a chip. What is further needed is a device that is made of a chain of chips. What is further needed is a device that is a chip or a chain of chips that can be implanted anywhere in the body. What is also needed is a device that has a power source that does not need to be replaced.

### SUMMARY OF INVENTION

The device such as the chip, also called BioMed-Chip, for electrical stimulation using RF energy harvesting of the present invention overcomes the disadvantages of the prior art, fulfills the needs in the prior art, and accomplishes its various purposes by providing a stimulation device that harvests radio frequency energy and is implantable in the human body. The chip device of the invention may include an electrode on one side that can provide neurostimulation and a harvesting side on the other. The chip device instead of the electrode may have a pouch containing a drug, a protein or a plasmid for localized delivery. In addition the chip could have the ability to transmit information to other chips in the body or create an RF field for other chips in the vicinity.

In accordance with one aspect of the invention, a chip for stimulation using radio frequency harvesting includes a circuit, the circuit comprising a radio frequency harvesting power circuit and a stimulation circuit, and a plurality of small electrodes coupled to the circuit, the plurality of electrodes provide stimulation to targeted areas of the body. An advantage of this system is that it may use “trickle charging” wherein the device is charged by the harvesting power circuit. Moreover, another advantage of this invention is the power transmitter which sends power to the device can be used both to send power and to send information. Further in accordance with another aspect of the invention through the use of RF filed generating chips in a stacked fashion going from under the skin to the site of stimulation enables such stimulations to occur deep in the human body. Such stacked chips can be used to transmit power and information from deep within the body.

There has been outlined, rather broadly, the more important features of the invention in order that the detailed description thereof that follows may be better understood, and in order that the present contribution to the art may be better appreciated. There are, of course, additional features of the invention that will be described below and which will form the subject matter of the claims appended herein.

In this respect, before explaining at least one embodiment of the invention in detail, it is to be understood that the invention is not limited in its application to the details of design and to the arrangement of components set forth in the following description or illustrated in the drawings. The invention is capable of other embodiments and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other methods and systems for carrying out the several purposes of the present invention. It is important, therefore, that the claims be regarded as including such equivalent methods and systems insofar as they do not depart from the spirit and scope of the present invention.

### BRIEF DESCRIPTION OF THE DRAWINGS

The accompanying drawings, which are incorporated in and form a part of the specification, illustrate the embodiments of the present invention and together with the description, serve to explain the principles of the invention. The chip devices 230, 300, 420, 530, 610, 900 and 1010 are the same type of radio frequency harvesting chip. The chip devices 220, 400, 410 and 620 are harvesting chips that can also generate an RF field. In the drawings:

FIG. 1 is a schematic representation of a prior art device for vagus nerve stimulation, 120 is the pacemaker power supply, 110 is the leads, 100 are the electrodes;

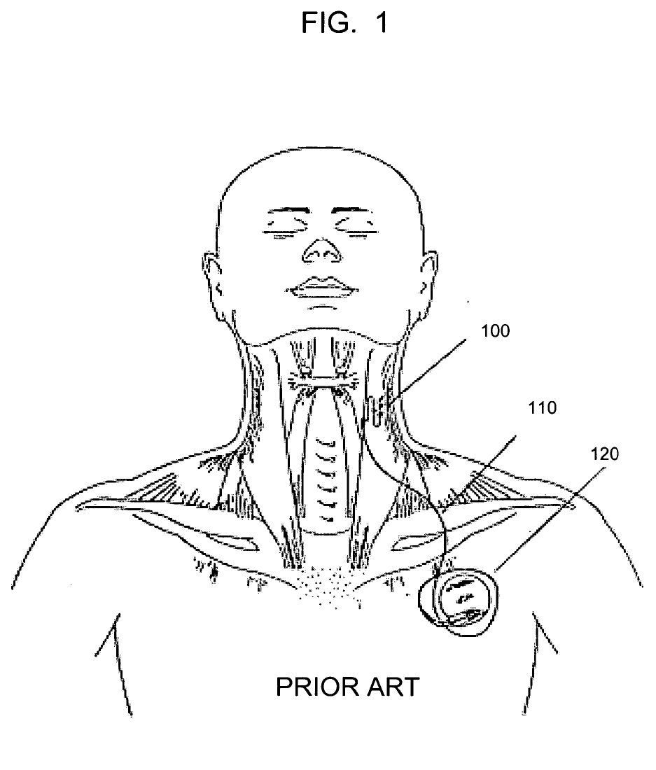

FIG. 2 is a schematic representation of a chip stimulation device using RF energy harvesting in accordance with the invention, 200 is the chip antenna, 210 is the chip microcontroller and 220 is the chip electrode;

FIG. 3 is a schematic representation of the chip device and its use in the human body using RF energy harvesting in accordance with the invention, 300 is a single harvesting or RF generating chip, 310 is a chain of chips for stimulation, 320 is a ring of chips for stimulation;

FIG. 4 is a schematic representation of the use of a chain of chip device for gastric electrical stimulation, 400 is the RF harvesting/generating chip that is located subcutaneously, 410 is RF harvesting/generating chip stacked in between 400 and the chain of stimulating chips (420), 400 and 410 power 420 in accordance with the invention;

FIG. 5 is a schematic representation of the chip device for localized drug delivery, 500 represents the drug chamber, 510 is the outside of the pouch containing the drug, 520 is the pore through which the drugs are eluted, 530 is the chip device;

FIG. 6 is a schematic representation of the chip stimulation device for vagus nerve stimulation in accordance with the invention; 600 is the vagus nerve, 610 is the ring of chips, 620 is the RF generating chips as described in FIG. 4;

FIG. 7 is a schematic representation of an external programming circuit in accordance with the invention;

FIG. 8 is a schematic representation of an alternative embodiment of the external power circuit non-inductively coupled to the power circuit in accordance with the invention;

FIG. 9 is an illustration of a band aid layout with a chip electrode that can be used on wounds to provide enhanced healing in accordance with the invention;

FIG. 10 is an illustration of the use of the chip electrode device (1000) for muscle stimulation using a wrist watch transmitter to power the chip in accordance with the invention.

### DETAILED DESCRIPTION OF THE INVENTION

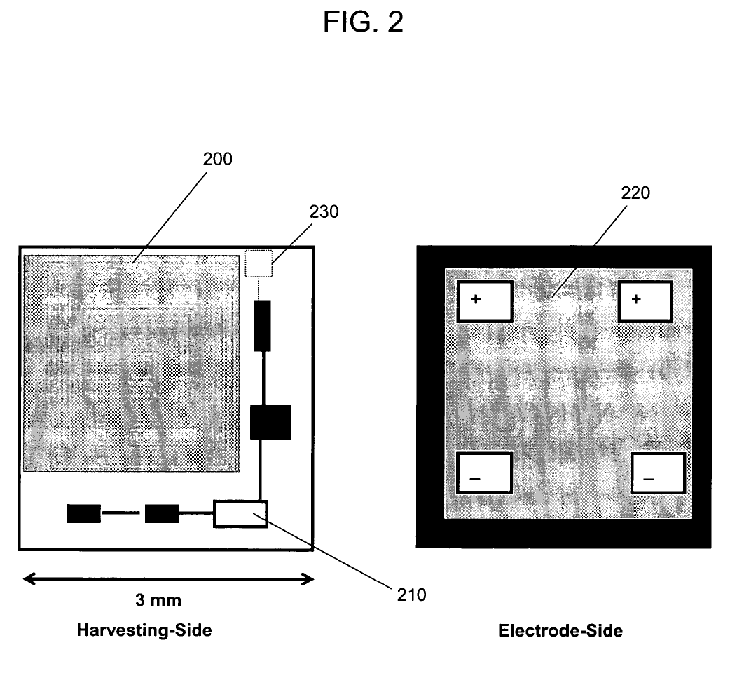

A device for stimulation using RF energy harvesting of the invention is called the BioMed-chip and is shown in FIG. 2. The BioMed-Chip consists of a flexible, implantable chip and antennae 200 having a dimension of approxiametly 9 mm² and a thickness of between 3 and 4 mm may be formed of a biocompatible material and include circuitry as further described herein. Electrodes 220 may include conventional electrodes or a flat electrode surface. The chip has a microcontroller 210. The circuitry may be operable to harvest and store RF energy, control the operation of the device and provide stimulation pulses and signals to the targeted areas of the body. The chip can be used singly or as a chain or ring of chips. In addition to harvesting RF certain BioMed-chips can also generate an RF field to power adjacent chips, or can transmit/ receive data from outside the body or to and from other chips inside the body. For such chips that can generate an RF field there is an RF generation/transmitting circuitry 230.

The BioMed-chip will include an antenna for harvesting or transmission, an electrode and a rechargeable storage device, either a thin film battery or capacitor that can be slowly charged over a period of time from the surrounding radio waves present in our environment.

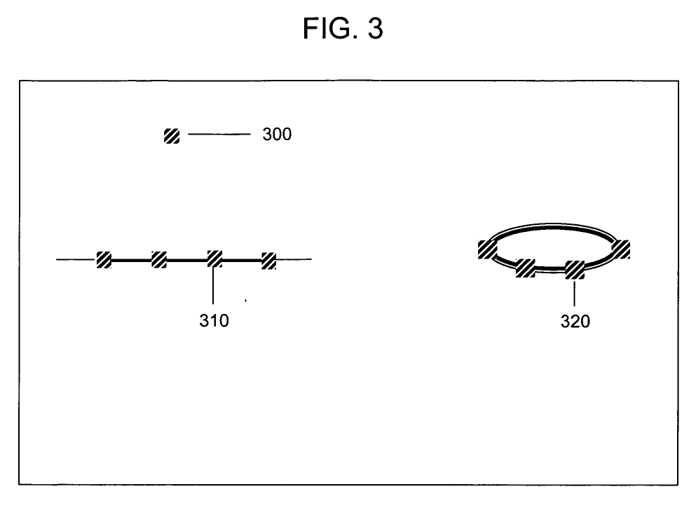

A device or “chip” for stimulation using RF energy harvesting of the invention is shown in FIG. 3. The flexible, implantable chip 300 can be used as a single chip or as a chain 310 or as a ring 320. Use of a chain of chips inside the body would require stacking of a series of RF generating chips to be placed all the way to under the surface of the skin (subcutaneous).

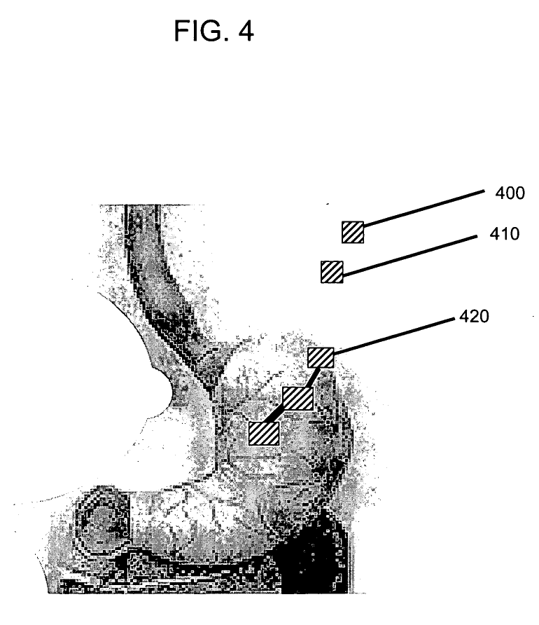

With reference to FIGS. 4 a chain of chips, 420 is put on the surface of the stomach and used to cause gasric electrical stimulation (GES). The electrodes are attached to the surface of the stomach. The surgery required for insertion of the chips on the stomach is fairly routine. A second (410), third (400) or more RF generating BioMed chips would be put in a stacked fashion upto the surface of the skin (subcutaneous) in order to power the chips on the surface of the stomach. The requirement for the RF-field generating chips would vary from patient to patient.

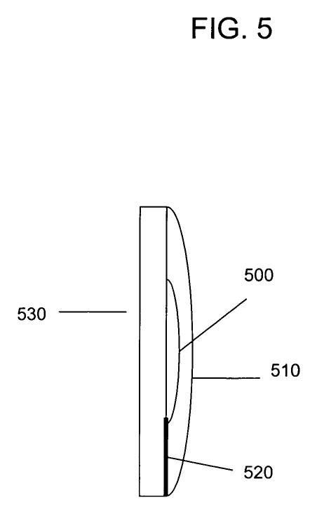

With reference to FIGS. 5 a pouch 510 on the back of the chip is used to store drug, proteins or plasmids. Stimulation of the pouch chamber 500 results in the release of the drug through the pore 520 or protein to the site at which the chip 530 is placed. The chip is implanted to a localized site such as the knee for rheumatoid arthritis where delivery of a drug or therapeutic protein is of value to the specific site. Implantation of the drug chip is done under local anesthesia and is accomplished easily.

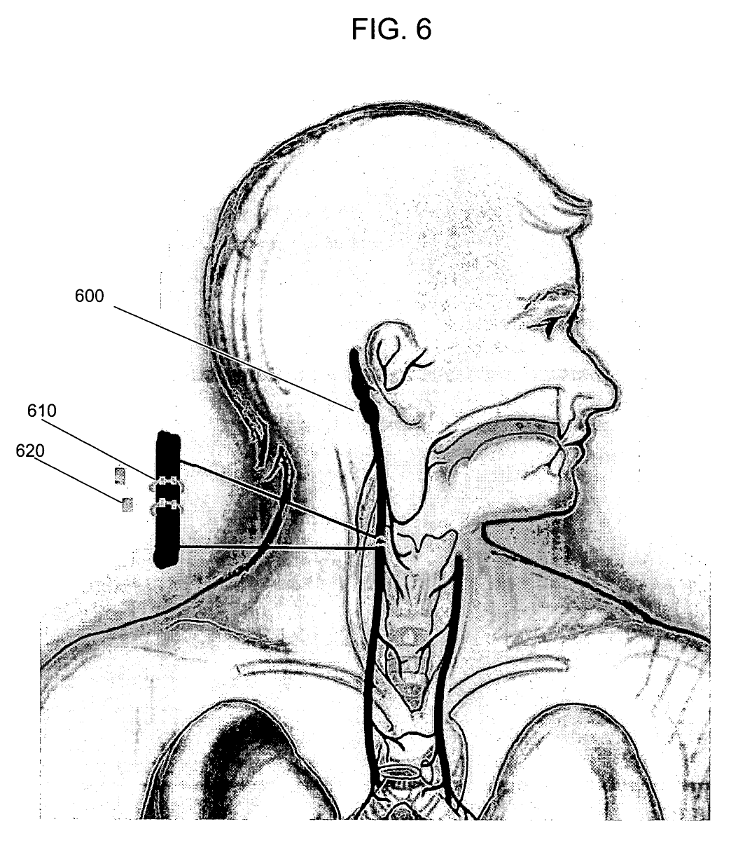

With reference to FIGS. 6 a ring of chips 610 are put on the the vagus nerve as shown in the figure. The ring of chips would be clipped on and anchored to the surrounding tissue to prevent migration of the ring. The use of one or more RF generating chips 620 more in a stacked fashion to under the skin surface (subcutaneous) is needed to power the ring of chips. The procedure is performed under general anesthesia. A neurosurgeon implants the ring of chips carefully attaching it to the vagus nerve. Implantation of the RF-field generating BioMed chip is then done. The number of chips needed would vary from patient to patient. Implantation is usually accomplished within 1 to 2 hours. For a few days following the procedure, the chips are programmed to stimulate the vagus nerve at regular intervals (e.g., for 30 seconds every 5 minutes) at a frequency determined by the doctor and patient. The physician adjusts the frequency using a computer.

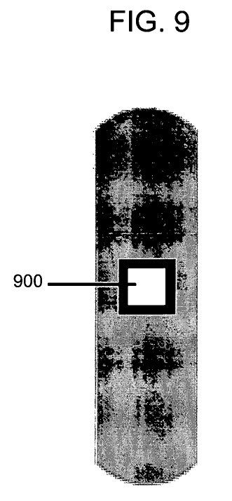

With reference to FIGS. 9 an example of a device for enhanced wound healing consists of a band aid with a Biomed chip 900 in the center. The mild electrical stimulation is delivered through the electrode surface which is in contact with the wound.

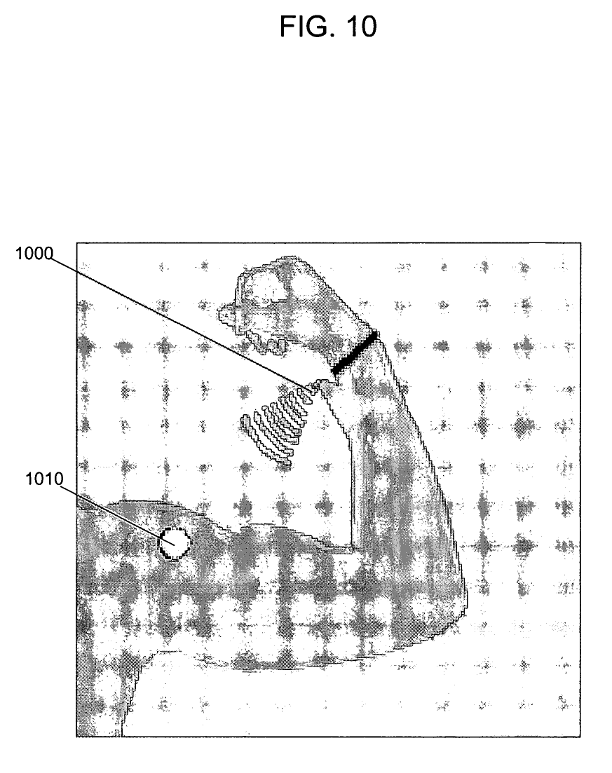

With reference to FIGS. 10 an example of a chip device for muscle stimulation with a Biomed chip 1010 the powering of the chip is done by an RF transmitter worn on a wrist watch 1000.

The chip circuitry may include a stimulation circuit as shown in FIG. 2 and a power circuit. The chip also has a microcontroller for a chip may manage the internal stimulation circuitry. A low frequency receiver may be coupled to the microcontroller and may convert RF communications into programming commands which the microcontroller interprets thus providing for ability to modify the chip functions.

According to internal parameters which can be modified via an external RF programming signal it should be possible to adjust the frequency output, varied voltage output and pulse duration. The microcontroller may be operated with an internal clock frequency of 125 KHz, giving an efficient tradeoff between power conservation and proper functionality. This clock frequency allows pulse durations in increments of 32 micro-seconds. The output pulse duration can be adjusted between ˜60 and ˜180 microseconds.

Every pulsing cycle, the programming input from the low frequency receiver chip 320 may be checked. If a programming signal is present, an input code may be read sequentially and the specified parameter adjusted to a new value, after which the program continues its pulsing routine.

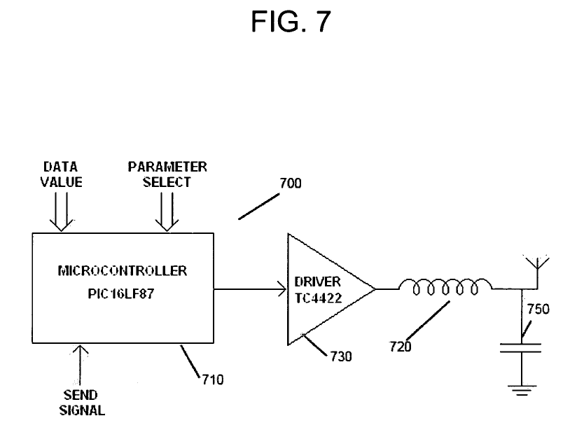

An external programmer circuit 700 may include a microcontroller 710 including a PIC16LF87, an inductor/capacitor (LC) oscillating circuit 720 (125 KHz), and an intermediate MOSFET driver 730 including a TC4422 as shown in FIG. 7. The MOSFET driver 730 may supply enough energy for driving the LC circuit 720. When a programming signal is to be sent, a button (not shown) may be pressed, telling the microcontroller 710 to read its inputs and stimulate the MOSFET driver 730 to oscillate the LC circuit 720 according to a communication protocol. Input voltages may be controlled by simple switches. Four switches may dictate the value to be sent, while five switches may dictate which parameter is to be changed. Only one of these switches should be on at one time. A Phidget RFID antenna 740 designed for 125 KHz may be attached to the high voltage side of a capacitor 750 of the LC circuit 720 for sending the programming signal. The circuit 700 may be powered via a 12-Volt wall supply. The 12 V drives the MOSFET driver 730 and is regulated to 5 V for the switches and microcontroller 710.

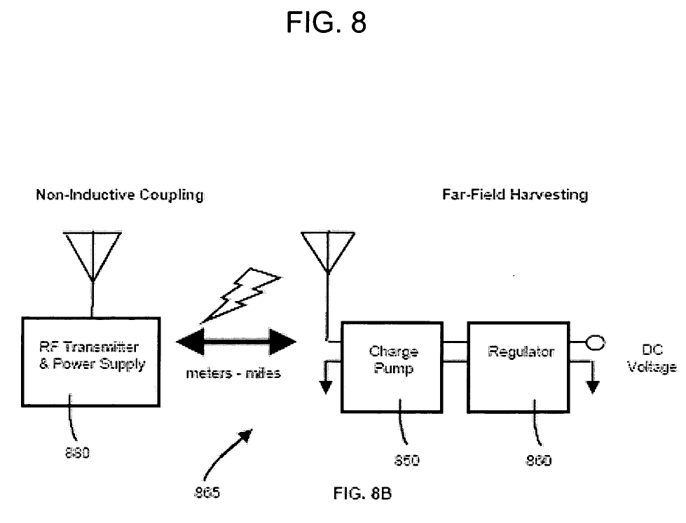

An external powering circuit 800 may in one embodiment described above provide for near field harvesting and includes inductive coupling between coils 835 and 840. With reference to FIG. 8 and in an alternative embodiment of the invention, the power circuit 865 for powering the stimulation circuit may be non-inductively coupled to an external source of RF energy 880. In this far field embodiment, the power circuit 865 may be disposed in a wrist band worn by the patient, in a room transmitter or in a transmitter disposed in a building occupied by the patient. In yet another alternative embodiment of the invention, the power circuit 865 may harvest ambient RF energy such as energy transmitted in space by using an inherently tuned antenna as described in U.S. Pat. No. 6,856,291, the description of which is incorporated by reference in its entirety herein. Furthermore, a rechargeable battery or other storage device (not shown) may be employed to store harvested energy. “Non-inductive” as described herein being directed RF.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

We claim:

1. A system for stimulation using radio frequency harvesting comprising:
a device implantable inside of a human body, the device comprising a radio frequency harvesting power circuit and a stimulation circuit mounted on a chip; and
at least one electrode coupled to the stimulation circuit, the one electrode on a chip configured to provid stimulation to targeted areas of the body.

2. The system of claim 1, wherein the implantable device is fabricated from a biocompatible substrate.

3. The system of claim 1, wherein the implantable device is a single chip, a chain of chips or a ring of chips.

4. The system of claim 1, wherein the radio frequency harvesting power circuit comprises a charge pump inductively coupled to a primary coil of an external power circuit.

5. The system of claim 1, wherein the radio frequency harvesting power circuit comprises an inherently tuned antenna for harvesting energy transmitted in space.

6. The system of claim 1, wherein the radio frequency harvesting power circuit comprises an RF transmitting chip in the human body.

7. The system of claim 1, wherein the radio frequency harvesting power circuit comprises RF transmitting chips stacked next to each other positioned from a skin surface to the radio frequency harvesting chip.

8. The system of claim 1, wherein the at least one chip electrode provides stimulation anywhere in the body.

9. The system of claim 1, wherein the at least one chip electrode is configured to provide vagus nerve stimulation.

10. The system of claim 1, wherein the at least one chip electrode is configured to provide gastric nerve stimulation.

11. The system of claim 1, wherein the at least one chip electrode is configured to provide localized drug delivery.

12. The system of claim 1, wherein the at least one chip electrode is configured to provide stimulation for facial nerve injury.

13. The system of claim 1, wherein the at least one chip electrode is configured to provide stimulation for unilateral paralysis.

14. The system of claim 1, wherein the at least one chip electrode is configured to provide stimulation of muscles in the body.

15. The system of claim 1, wherein the at least one chip electrode is configured to provide stimulation for relief of pain.

16. The system of claim 1, further comprising a programming circuit operable to control the stimulation circuit.

17. The system of claim 16, wherein the programming circuit is operable to control a stimulation circuit voltage output.

18. The system of claim 16, wherein the programming circuit is operable to control a stimulation circuit output pulse width.

19. The system of claim 16, wherein the programming circuit is operable to control a stimulation circuit output frequency.

20. The system of claim 1, further comprising an energy storage device coupled to the implantable device.

21. The system of claim 1, whereinat least one chip electrode is configured to provide stimulation to treat depression.

22. The system of claim 1, wherein the at least one chip electrode is configured to provide stimulation to treat Bell's Palsy.

23. The system of claim 1, wherein the at least one chip electrode is configured to provide stimulation to treat obesity.

24. The system of claim 1, wherein the at least one chip electrode is configured to provide stimulation to treat Alzheimer's disease.

25. A system for stimulation using radio frequency harvesting comprising:
a first implantable chip inside of a human body, the first implantable ship comprising a radio frequency harvesting power circuit and a stimulation circuit; and
a second implantable chip inside of a human body, the second implantable ship comprising a radio frequency harvesting power circuit and a stimulation circuit, wherein the first implantable chip generates radio frequency energy for harvesting by the second implantable chip.

26. The system of claim 25, further comprising:
a third implantable chip inside of a human body, the third implantable ship comprising a radio frequency harvesting power circuit and a stimulation circuit, wherein the second implantable chip generates radio frequency energy for harvesting by the third implantable chip.

27. The system of claim 25, wherein the first implantable chip is harvests radio frequency energy from an external radio frequency transmitter.

28. The system of claim 25, wherein the first implantable chip is positioned under a skin surface, and the second implantable chip is positioned deeper in a patient.
